(19) United States
(12) Patent Application Publication
(10) Pub. No.: US 2018/0369140 A1
(43) Pub. Date: Dec. 27, 2018

(54) NANOVECTORS FOR PENETRATING BRAIN TUMOR TISSUES TO CONDUCT GENE THERAPY

(71) Applicant: University of Houston System, Houston, TX (US)

(72) Inventors: Zhifeng REN, Houston, TX (US); Dong CAI, Houston, TX (US); Jay-Jiguang ZHU, Houston, TX (US)

(21) Appl. No.: 16/029,809

(22) Filed: Jul. 9, 2018

Related U.S. Application Data
(63) Continuation of application No. 15/583,274, filed on May 1, 2017, now abandoned.
Application No. 15/583,274 is a continuation-in-part of application No. PCT/US2015/057828, filed on Oct. 28, 2015.
(60) Provisional application No. 62/072,125, filed on Oct. 29, 2014.

(51) Int. Cl. A61K 9/00; A61K 47/69; A61K 48/00; A61K 33/24; A61K 31/713; A61K 31/555; A61K 31/519; A61K 31/495; A61K 31/4745; A61K 31/175; A61K 41/00; A61K 31/337; A61K 9/50

(52) U.S. Cl. CPC A61K 9/0092; A61K 47/6925; B82Y 5/00; A61K 33/24; A61K 31/713; A61K 31/555; A61K 31/519; A61K 31/495; A61K 31/4745; A61K 31/175; A61K 41/00; A61K 31/337; A61K 9/5094; A61K 48/0083; A61K 48/0058

(57) ABSTRACT

A method of selectively targeting a cell with a therapeutic agent, the method comprising: targeting a cell with a nanospear, puncturing the cell with said nanospear; releasing a therapeutic agent from said nanospear, wherein said therapeutic agent enters said cell, thereby effecting the efficacy of said cell.

### CROSS-REFERENCE TO RELATED APPLICATIONS

This application is a continuation of U.S. patent application Ser. No. 15/583,274, filed May 1, 2017, which claims priority to PCT/US2015/057828, filed Oct. 28, 2015, which claims priority to U.S. Provisional Patent Application No. 62/072,125, filed Oct. 29, 2014, the content of each being hereby incorporated by reference in their entirety for all purposes.

### STATEMENT REGARDING SPONSORED RESEARCH

Not applicable.

### TECHNICAL FIELD

This disclosure relates to methods of selectively targeting cells with a therapeutic agent. More specifically, it relates to methods of targeting cells, tumors, and solid tumors using nanospears comprising magnetized carbon nanotubes and therapeutic agents. Even more specifically this disclosure relates to methods of using a non-viral gene vector to treat a solid tumor (such as glioblastoma), wherein a nanospear is employed to deliver the vector directly to the cellular target.

### BACKGROUND

Glioblastoma (GBM) is an incurable form of brain cancer with poor prognosis. Current treatments of GBM have a low success rate due to a number of reasons including: the non-specific cell toxicity of current treatments, radio exposure of healthy cells, insufficient transport of drugs across blood-brain-barrier, heterogenic GBM of the tumor, and the highly infiltrative nature of GBM cells. Current drug delivery approaches typically leave drug molecules to passively diffuse to a target after their release into the subject, therefore resulting in sub-optimal delivery to deep and solid tissues, such as is typical in GBM deep tissue tumors. Viral vectors (that are used to deliver genetic material into cells) display high delivery efficiency but exhibit no target selectivity in their infection and may trigger immunogenic responses and oncogenesis concerns. Therefore a method of selectively and efficiently targeting deep or solid tumors, such as but not limited to GBM, is an unmet need in the art.

### BRIEF SUMMARY

In an effort to address such unmet needs as described above, disclosed herein, is a non-viral gene therapy vector. In some embodiments the non-viral gene therapy vector may function with the efficiency of viral infection. In some embodiments herein disclosed is a therapeutic composition comprising: a nanospear, wherein the nanospear comprises: a carbon nanotube (CNT); a magnetic particle; and a therapeutic agent, wherein the nanospear comprises a polymer coating, wherein the coating comprises said therapeutic agent, in some embodiments the therapeutic agent is selected from the group comprising: Temozolomide, BCNU, Irinotecan, Carboplatin, Cisplatin cpt-11, Taxol, Methotrexate, a non-viral gene vector, or combinations thereof, in other embodiments the therapeutic agent is a non-viral gene vector that comprises a transgene plasmid, wherein the plasmid comprises miRNA-124 target sites that suppress the mRNA of the anti-neural function protein SCP1 (small C-terminal domain phosphatase 1) In some embodiments, the therapeutic composition comprises a non-viral gene vector which comprises a transgene plasmid, wherein said plasmid further comprises miRNA-124 target sites.

In another embodiment, a method of selectively targeting a tumor with a therapeutic agent is described, the method comprises (a) targeting the tumor with a nanospear; wherein the nanospear is coated with a polymer and wherein the polymer encapsulates a therapeutic agent, the nanospear further comprises a magnetic particle and a block chain linker, wherein the block chain linker bonds the therapeutic agent to the polymer; (b) sensing the presence of the tumor; wherein sensing is by biorecognition of enzymatic activity associated with tumorigenesis; c) enzymatically degrading the block chain linker; wherein the tumor comprises an enzyme that degrades the block chain linker; and (c) releasing the therapeutic agent from the nanospear, wherein the agent is selective for the tumor. In another embodiment the tumor is comprised of tumor tissue; blood vessels that surround the tumor tissue; and tumor cells. In some further embodiments, the nanospears concentrate within the tumor; and in a still further embodiment the nanospears after step (a) further penetrate the tumor. In another embodiment of selectively targeting a tumor, the enzyme in step (c) comprises a matrix metalloproteinase 2, a metalloproteinase 9, or a combination thereof; in some embodiments the therapeutic agent is selected from one of more chemotherapy drugs to treat heterogenetic tumor cells. In a further embodiment the nanospear penetrates at least a first layer of tumor cells.

In some embodiments of the method of selectively targeting a tumor with a therapeutic agent, the therapeutic agent is: a drug molecule; a non-viral gene therapy vector, a molecule that reduces the growth of said tumor; a molecule that induced apoptosis, an imaging agent, a molecule that inhibits growth of said tumor; or a combination thereof. In other embodiments the tumor is a solid tissue tumor, and in a further embodiment, the tumor is glioblastoma (GBM). In some embodiments of the method of selectively targeting a tumor with a therapeutic agent the targeting comprises subjecting the nanospear is a magnetic force, in a further embodiment the force is by produced by a Halbach magnet.

In one embodiment disclosed herein, is a therapeutic method of treating a subject, wherein the subject comprises a tumor, and the method comprises administering to the subject a nanospear, subjecting the nanospear to a magnetic force, wherein the magnetic force guides the nanospear, localizing the nanospear at the tumor, penetrating the tumor, and releasing a therapeutic agent from the nanospear. In some embodiments, administering is by intravenous injection or subcutaneous injection.

In one embodiment disclosed herein, is a method of selectively targeting a cell with a therapeutic agent, the method comprising: targeting a cell with a nanospear, puncturing the cell with the nanospear; releasing a therapeutic agent from the nanospear, wherein the therapeutic agent enters the cell and thereby effecting the growth of the cell. In some embodiments described herein, the cell comprises: a multilayer cell culture, a 3D neuron cultures, a spheroid, a GBM tumor tissue, or combinations thereof. In some embodiments, effecting the growth of a cell comprises at least one of inducing cell growth stasis or inhibition, inhibition of molecular pathways, cellular mechanisms, or by cell death/apoptosis.

The foregoing has broadly outlined certain features exemplary embodiments of the present disclosure in order that the detailed description that follows may be better understood. It should be appreciated by those skilled in the art that the conception and the specific embodiments disclosed may be readily utilized as a basis for modifying or designing other methods and structures for carrying out the same purposes of the disclosure as claimed below. It should also be realized by those skilled in the art that such equivalent constructions do not depart from the spirit and scope of the disclosure as set forth in the appended claims.

### BRIEF DESCRIPTION OF THE DRAWINGS

For a more complete understanding of the present disclosure, reference will now be made to the accompanying figures:

FIG. 1 (A-G) illustrates an embodiment of surface modification and characterization of CNTs as described herein. FIG. 1A shows a schematic illustration of the surface modification of CNTs: wherein Ni-coat CNTs array by e-beam evaporation of Ni on an aligned CNTs array, and poly-_{L}-tyrosine coating by electropolymerization. FIG. 1B depicts recording of cyclic voltammetry (CV) for electropolymerization of _{L}-tyrosine on CNTs, with CNTs and Ag/AgCl as the working and reference electrodes, respectively. FIG. 1C shows deposition charge (Q) by integration of each cycle of CV versus the cycles. (D) SEM image of Ni-coated CNTs. FIG. 1E shows TEM images of Ni-coated CNTs with surface modified by poly-_{L}-tyrosine coating, as indicated by the red arrow; inset: a low magnification image. FIG. 1F shows magnetization measurement of Ni-coated CNTs. FIG. 1G shows aqueous suspension of the magnetized CNTs.

### DETAILED DESCRIPTION OF THE DISCLOSURE

The following discussion is directed to various exemplary embodiments of the disclosure. However, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and that the scope of this disclosure, including the claims, is not limited to that embodiment.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct engagement between the two devices, or through an indirect connection via other intermediate devices and connections. As used herein, the term “about,” when used in conjunction with a percentage or other numerical amount, means plus or minus 10% of that percentage or other numerical amount. For example, the term “about 80%,” would encompass 80% plus or minus 8%.

In some embodiments described herein, a nanospearing methodology is disclosed, wherein a gene-bearing nanospear structure may be injected into a target, wherein the target may be isolated cells in vitro, or in a subject (or patient) and in vivo. The administration of such nanospears may therefore be intravenous or subcutaneous. The nanospears comprise magnetized carbon nanotubes that are coated with a biocompatible polymer that may be linked or chemically bonded or attached to a therapeutic agent. The nanospear may be guided by a magnetic source to a cellular target such as GBM, where the specific localized tumor environment may induce cleavage of the linker between the polymer and the therapeutic agent, thereby delivering the agent directly and specifically to the localized target cells that comprise for example a GBM tumor.

In some embodiments the nanospears will be concentrated at the blood vessels surrounding the tumor tissue, and will further spear, and may penetrate into the cells of the tumor tissue itself. Such spears, in some embodiments therefore penetrate the cells of the tumor tissue layer by layer. In some embodiments Gene therapy molecules are disseminated into such cells, in some embodiments the trajectory of the nanospear and penetration of the cells within that trajectory facilitates the dissemination of the gene therapy molecules into the cells.

### EXAMPLES

Production of nanospears: In some embodiments the nanospears disclosed herein comprise: biocompatible and biodegradable materials (coating polymers), iron oxide magnetic nanoparticles, carbon nanotubes and therapeutic agents. Therapeutic agents delivered by the nanospears include but are not limited to chemotherapy drugs, for example those selective for GBM, such as Temozolomide, BCNU, Irinotecan, Carboplatin, Cisplatin cpt-11, Taxol, Methotrexate. The drugs (therapeutic agents) are entrapped in a polymer coating outside the magnetic bmCNT. (carbon-nanotube). In some embodiments the coating on the carbon nanotube may be produced by electropolymerization, or self-assembly by static electrical charge, or by micelle interaction. Examples of the polymer coating include (but are not limited to) polyphenol, polytyrosine, polyaniline, and polypyrrole. In some embodiments, the polymer used for drug encapsulation of the nanospear may be designed to response to a specific environmental change that is related only to cancer, so that the drug may be released only around the cancer target. For example, in some embodiments, a block polymer that is sensitive to the cancer related enzymatic activity maybe incorporated in the nanostructures to render such a feature of targeted release. In some embodiments a chemical linker in the form of a block chain linker links the polymer coating to the therapeutic agent, when the nanospear comes into contact with matrix metalloproteinases found in the vicinity of GMB tumors, matrix metalloproteinase 2, a metalloproteinase 9, or a combination thereof, digest the linker and release the therapeutic agent, thereby allowing delivery of the agent to the target tumor cell. Examples of block chain linkers include but are not limited to KRGPQGIWGQDRCGR (Seq. 1), KRGPQGIAGQDRCGR (Seq. 2), KRGDQGIAGFDRCGR (Seq. 3) and GPQGIFGQ (Seq. 4).

In some embodiments, nanospears comprise carbon nanotubes (CNTs), a magnetic metal and an outer polymeric layer. In an embodiment, the magnetic metal may comprise magnetic particles and a magnetic metal layer. In an embodiment, the magnetic metal may comprise nickel, Iron (Fe), Iron oxide, superparamagnetic materials, and the like, or combinations thereof.

In an embodiment, the CNTs have a rod shape or cylindrical geometry. In such embodiment, the CNTs may be characterized by having two ends, which correspond to the ends of the rod or cylinder. Further, in such an embodiment, the magnetic metal may coat only one end of the nanospears. Coating only one end of the CNTs with a magnetic material such as, magnetic metal) ensures that the resulting nanospears could be oriented in the magnetic field, and could consequently be “speared” in the desired direction. For purposes of the disclosure herein, the terms “spear” or “spearing,” and “nanospear” or “nanospearing,” may be used interchangeably and all these related terms refer to a directed movement of a magnetized nanostructure (MNS) within and/or through a bioentity (such as, a single cell, distinct cell layers, a clump of cells, a piece of live tissue, etc.). Non-limiting examples of MNS include nanospear, nanotube, nanoparticle, nanorod, nanowire, nanohorn, nanostar, nanovesicle, nanocapsule that may comprise inorganic, organic, polymeric, metallic, non-metallic, oxide, alloy, or composite materials, and the like, or combinations thereof.

In some embodiments, the nanospears may be characterized by a nanospear length of from about 0.5 mm to about 5 mm, alternatively from about 1 mm to about 3 mm, or alternatively from about 1 mm to about 2 mm.

In an embodiment, the nanospears may be characterized by a nanospear diameter of from about 50 nm to about 300 nm, alternatively from about 75 nm to about 200 nm, or alternatively from about 75 nm to about 125 nm.

In an embodiment, a method of preparing nanospears may comprise growing carbon nanotubes; coating the carbon nanotubes with a magnetic metal to yield nanospears, wherein the magnetic metal may comprise nickel; and coating the nanospears with an outer polymeric layer, wherein the outer polymeric layer may be hydrophilic and biocompatible.

In some embodiments, the CNTs may be grown by using any suitable methodology (such as for example those disclosed in U.S. Provisional Patent application 62/032,996 incorporated herein in its entirety). In an embodiment, the CNTs may be grown by using a plasma-enhanced chemical vapor deposition system, as described in more detail in science 1998, 282(5391):1105-1107 (27), which is also incorporated by reference herein in its entirety. The growth of the CNTs may result in straight-aligned CNTs with magnetic nickel (Ni) particles enclosed at the tips (or in further embodiments as described herein with Iron, or Iron oxide enclosed at the tips) which make the CNTs magnetically drivable. In an embodiment, a layer of a magnetic metal (such as, Ni or Fe) may be deposited along the surface of individual CNTs by using any suitable methodology, such as for example e-beam evaporation. In an embodiment, the layer of magnetic metal may enhance the magnetization, thereby leading to an enhanced magnetic force, wherein such magnetic force may be required for cell penetration. The magnetic metal may exacerbate toxicity and hydrophobicity of the nanospears for biological applications. In some embodiments the force required to to attract the nanospear to its target is between 0.01 pN and 1000 nN, in other embodiments the force required to attract the nanospear to its target is between 100 pN and 10 nN, and in further embodiments the force required to attract the nanospear to its target is between 1000 pN and 1 nN. In some embodiments the force required to attract the nanospear to its target may vary with respect to the types of cells and tissues that comprise the target, and in other embodiments the force may vary because of the type of magnetism and geometry of the nanospear. In other embodiments the Tesla range for the Halbach magnet is between 0.01 to 10 T, and in other embodiments the Tesla range is between 0.05 to 5 T. In some embodiments the field of magnet composing the Halback array is 1 to 5 T, and in further embodiments the field produced by the array in the center of the ring is 0.05 to 1 T. In an embodiment, the magnetic metal layer may be characterized by a magnetic metal layer thickness of from about 5 nm to about 50 nm, alternatively from about 10 nm to about 30 nm, or alternatively from about 15 nm to about 25 nm.

In an embodiment, the nanospears (such as, nanospears array) may be further coated with the outer polymeric layer by using any suitable methodology, such as for example electropolymerization, thereby reducing the toxicity of metal (such as, Ni)-coated CNTs. In such embodiment, the outer polymeric layer may comprise poly-l-tyrosine. In an embodiment, the outer polymeric layer may be hydrophilic, thereby rendering the nanospears hydrophilic. In an embodiment, the outer polymeric layer may be biocompatible, thereby rendering the nanospears biocompatible.

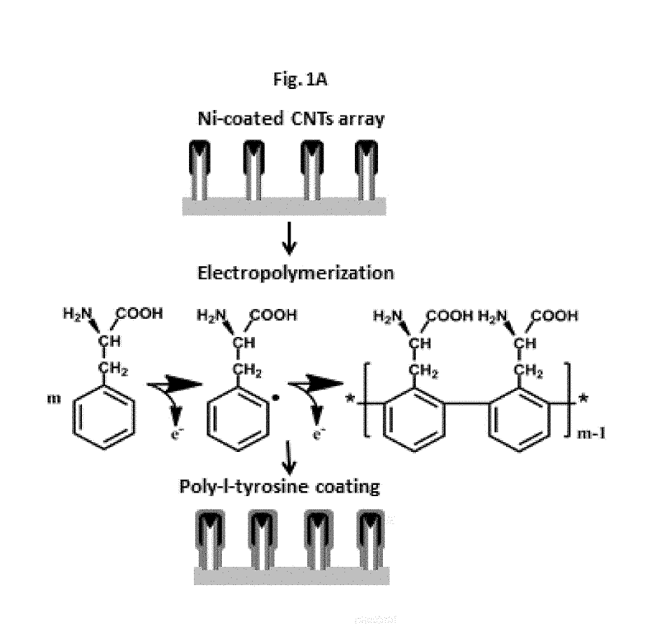

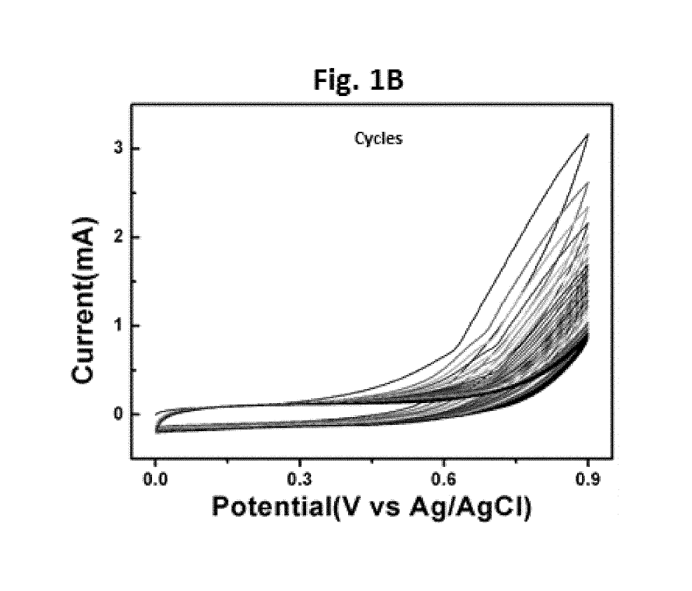

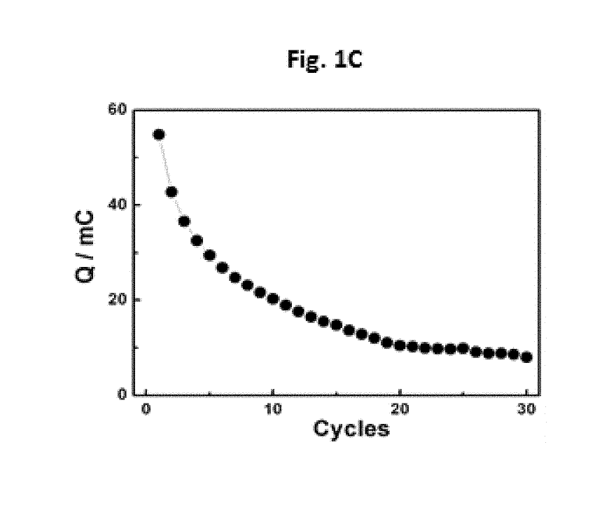

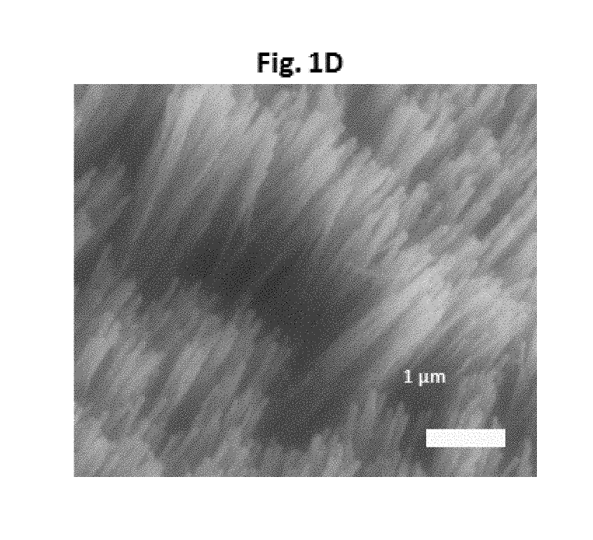

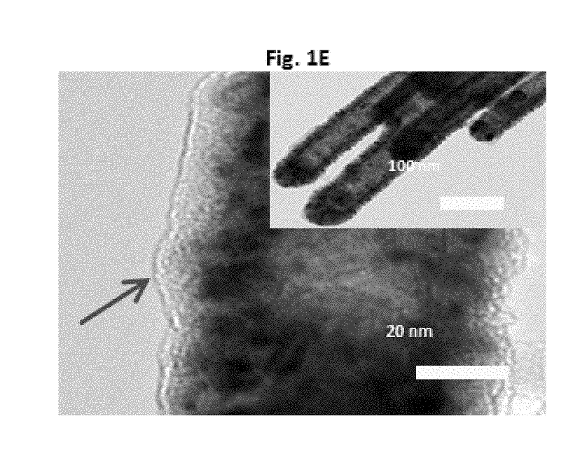

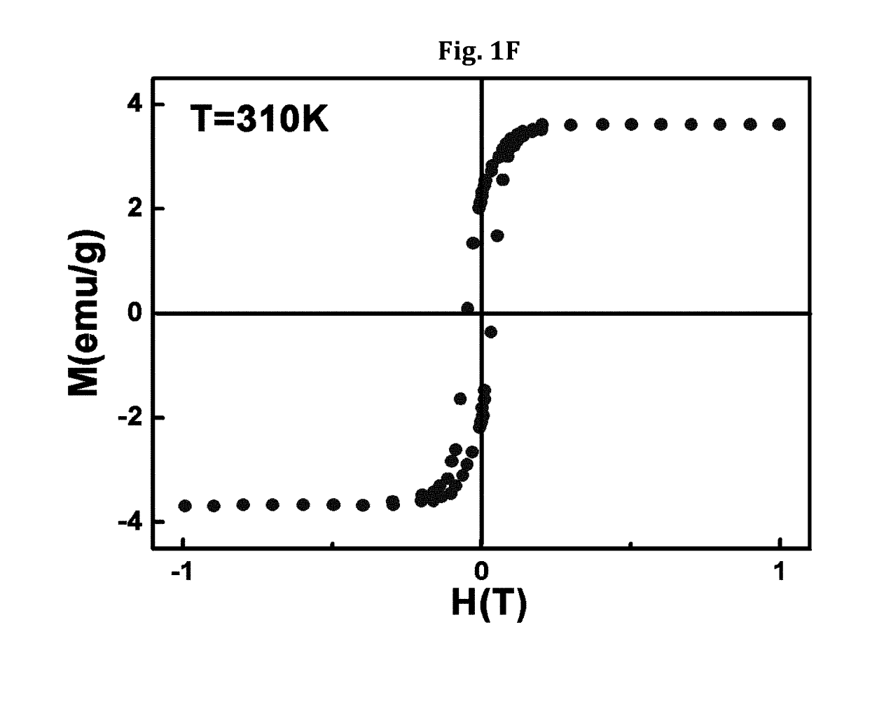

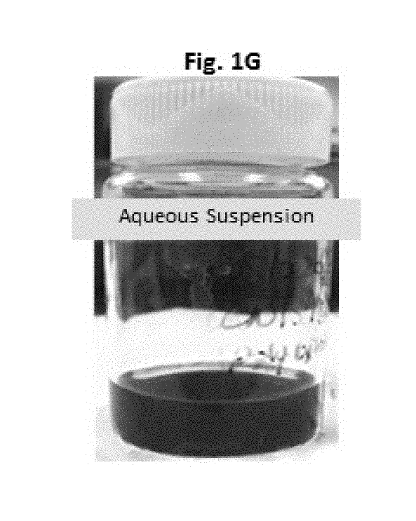

In an embodiment, the outer polymeric layer may be characterized by an outer polymeric layer thickness of from about 1 nm to about 50 nm, alternatively from about 2 nm to about 25 nm, or alternatively from about 5 nm to about 15 nm. In an embodiment, the nanospears (such as, nanospears array) may be connected to an electrochemistry system to conduct electropolymerization of a monomer (such as, ₗ-tyrosine) on the surfaces of the nanospears, as illustrated in FIG. 1. It has been previously shown that electropolymerization of ₗ-tyrosine may be a feasible way to create a hydrophilic and biocompatible film that is suitable in diverse biological applications, as described in more detail in biomacromolecules 2005, 6(3):1698-1706 and anal biochem 2009, 384(1):86-95 (28, 29), each of which is incorporated by reference herein in its entirety. In an embodiment, electropolymerization of ₗ-tyrosine into poly-ₗ-tyrosine may comprise cyclic voltammetry.

### Nanospear Targets:

In vitro targets may be prepared for analysis of the therapeutic effects and compositions of embodiments of the nanospears herein provided. For example, brain tumor cells may be enzymatically digested and dispersed in a petri dish and maintained in a CO₂ incubator at 37° C., under controlled CO₂ concentration and saturated humidity. The mono-dispersed cells may be re-digested and re-suspended, and transferred to 3D culture hydrogel. Some embodiments herein described use Mebiol Gel (Cosmo Bio Co., Ltd), which is liquidized poly(N-isopropylacrylamide) and poly(ethylene glycol) hydrogel in a cell culture medium on ice; the cells are then mixed with the hydrogel at low temperature (2-10° C.); (3) the hydrogel was warmed to 37° C. to solidify the hydrogel and maintain the cell in 3D scaffold. In some embodiments the resultant 3D culture environment provides conditions for cell proliferation, cell communication, gas and mass exchange, and maintains the specific location of the cells. The hydrogel may be kept in a cell culture plate or petri dish, wherein the cancer may be produced in days. Epithelial cells may be cultured as a non-cancer cell control to evaluate the selectivity of the targeted release. Further, in some embodiments described herein the environmental selectivity and stability of the polymeric nanospear may be measured in the artificial environment provided by buffer, or in the cultured normal and cancer cells. In some embodiments, the drug-bearing bmCNT, i.e. nanospears described herein were suspended in culture medium and applied to the hydrogel containing the spheroids. A magnet (Halbach) may be placed under the container (comprising the spheroids) to pull the nanospears into the hydrogel and the spheroid, wherein drug molecule may be released directly into the target cell (such as the solid tumor in vivo). Fluorescent molecules may be used instead of therapeutic agents to further analyze the progress of nanospear motion in the hydrogel visualized with a confocal microscope. In some embodiments the pharmacokinetics of the targeted release of the therapeutic agents from the nanospears may be evaluated by measuring fluorescence leakage of fluorescent surrogate under artificial environment with TIRF microscopy. The drugs molecules may be loaded into the nanospears at different molar concentrations such as but not limited to: 1 μM, 10 μM, 100 μM, 1 mM, 10 mM, 20 mM, 40 mM, and the cells may be maintained in the hydrogel for a selected time periods without disturbance for 0.5, 1, 2, 4, 8, 16, 24, 48, 72 hours for example.

The cells may be harvest into the liquid medium by cooling to 2-10° C. With this hydrogel feature, the dose response and time lapse of response may be obtained by propidium iodide staining. In further embodiments the nanospear may be electrospun, comprise nanofibers or nanobeads and may be produced with the polymers, drugs and magnetic particles described herein.

In some embodiments, nanospears may be administered in vivo or in vitro to selectively conduct GBM suppression by nanospear gene vector. The transgene plasmid may comprise miRNA-124 target sites, so that transgene expression is prevented in neuron, rather in glia. In some embodiments a mixed culture of neuron and glial in the form of cell layers, spheroids may be produced, thereby characterizing the vector dosage based on the viability of glial cells and neurons. In some embodiments a xenograft animal model may be utilized along with I.V. administration to assess the shrinkage of tumor size. Some embodiments of the method herein described showed high efficient plasmid transfection in cultured primary cortical neurons, and in further embodiments biocompatible nanospears thoroughly penetrated mammalian cell bodies

While exemplary embodiments of the disclosure have been shown and described, modifications thereof may be made by one skilled in the art without departing from the spirit and teachings of those embodiments. The embodiments described herein are exemplary only, and are not intended to be limiting. Many variations and modifications of the disclosed embodiments are possible and are within the scope of the claimed disclosure. Where numerical ranges or limitations are expressly stated, such express ranges or limitations should be understood to include iterative ranges or limitations of like magnitude falling within the expressly stated ranges or limitations (such as, from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the embodiments of the present disclosure. The disclosures of all patents, patent applications, and publications cited herein are hereby incorporated by reference, to the extent that they provide exemplary, procedural or other details supplementary to those set forth herein.

What is claimed is:

1. A method of selectively targeting a tumor with a therapeutic agent, the method comprising:
(a) targeting said tumor with a nanospear; wherein said nanospear comprises: a carbon nanotube (CNT) coated with a polymer, wherein said polymer encapsulates a therapeutic agent, and wherein said therapeutic agent and said polymer are linked by a block chain linker, and wherein said nanospear comprises a magnetic particle;
(b) sensing the presence of the tumor;
(c) enzymatically degrading said block chain linker; wherein said tumor comprises an enzyme that degrades said polymer; and
(d) releasing said therapeutic agent from said nanospear, wherein said agent is selective for said tumor.

2. The method of claim 1 wherein said tumor is comprises tumor tissue; blood vessels that surround said tumor tissue; and tumor cells.

3. The method of claim 1, wherein said nanospears concentrate within said tumor.

4. The method of claim 1, wherein said nanospears after step (a) further penetrate said tumor.

5. The method of claim 1, wherein said enzyme in step (c) comprises a matrix metalloproteinase 2, a metalloproteinase 9, or a combination thereof.

6. The method of claim 1, wherein said therapeutic agent is selected from one of more chemotherapy drugs that target heterogenetic tumor cells.

7. The method of claim 6, wherein said therapeutic agent is selected from the group comprising: Temozolomide, BCNU, Irinotecan, Carboplatin, Cisplatin cpt-11, Taxol, Methotrexate, a non-viral gene vector, or combinations thereof.

8. The method of claim 7 wherein said non-viral gene vector comprises a transgene plasmid, wherein said plasmid comprises miRNA-124 target sites.

9. The method of claim 1, wherein said nanospear penetrates at least a first layer of tumor cells.

10. The method of claim 1, wherein said therapeutic agent is a drug molecule; a non-viral gene therapy vector, a molecule that reduces the growth of said tumor; a molecule that induced apoptosis, an imaging agent, a molecule that inhibits growth of said tumor; or a combination thereof.

11. The method of claim 1, wherein said tumor is a solid tissue tumor.

12. The method of claim 11, wherein said tumor is glioblastoma (GBM).

13. The method of claim 1, wherein said targeting comprises subjecting said nanospear to a magnetic force.

14. The method of claim 13, wherein said force is by produced by a Halbach magnet.

15. A therapeutic method of treating a subject, wherein the subject comprises a tumor, said method comprising:
administering to said subject a nanospear;
subjecting the nanospear to a magnetic force, wherein said magnetic force guides said nanospear;
localizing said nanospear at said tumor;
penetrating said tumor; and
releasing a therapeutic agent from said nanospear.

16. The method of claim 15, wherein administering is by intravenous injection or subcutaneous injection.

17. A method of selectively targeting a cell with a therapeutic agent, said method comprising:
targeting a cell with a nanospear,
puncturing said cell with said nanospear;
releasing a therapeutic agent from said nanospear, wherein said therapeutic agent effects cell growth.

18. The method of claim 1, wherein said cell comprises: a multilayer cell culture, a 3D neuron cultures, a spheroid, a GBM tumor tissue, or combinations thereof.

19. The method of claim 17, wherein effects cell growth is by at least one of: inducing cell stasis, cell death, or inhibition of cellular mechanisms.


SEQUENCE LISTING (4 sequences)

SEQ ID NO: 1 - protein, 15 aa, Artifical
  Synthetic
KRGPQGIWGQDRCGR

SEQ ID NO: 2 - protein, 15 aa, Artifical
  Synthetic
KRGPQGIAGQDRCGR

SEQ ID NO: 3 - protein, 15 aa, Artificial
  Synthetic
KRGDQGIAGFDRCGR

SEQ ID NO: 4 - protein, 8 aa, Artificial
  Synthetic
GPQGIFGQ